(19) United States
(12) Patent Application Publication
(10) Pub. No.: US 2022/0110587 A1
(43) Pub. Date: Apr. 14, 2022

(54) BODY MOTION ANALYSIS DATA TREATMENT

(71) Applicant: LUXEMBOURG INSTITUTE OF SCIENCE AND TECHNOLOGY (LIST), Esch-sur-Alzette (LU)

(72) Inventor: Foued MELAKESSOU, Russange (FR)

(21) Appl. No.: 17/418,910

(22) PCT Filed: Dec. 20, 2019
(86) PCT No.: PCT/EP2019/086795

§ 371 Date: Jun. 28, 2021

(30) Foreign Application Priority Data
Dec. 28, 2018 (LU) LU101075

(51) Int. Cl. A61B 5/00; G06V 40/20; A61B 5/103; A61B 5/11

(52) U.S. Cl. CPC A61B 5/6807; G06V 40/23; A61B 5/1038; A61B 5/1112; A61B 2503/10; A61B 2505/09; A61B 5/112

(57) ABSTRACT

A method for monitoring a human body motion comprising the steps of: providing a garment with a surface comprising a plurality of stress sensors measuring at least one component of a stress tensor and generating output signals, the plurality of stress sensors being distributed over the surface and being adapted to face a portion of a human body; providing a means for detecting or recording 3D coordinates of each of the stress sensors; and estimating at least one stress value representative of a human body motion parameter in a point of the surface at least on the basis of at least two of the at least one components, corresponding respectively to the measurements of at least two of the stress sensors and at least two 3D coordinates, corresponding to the at least two of the stress sensors.

### CROSS-REFERENCE TO RELATED APPLICATIONS

The present invention is the US national stage under 35 U.S.C. § 371 of International Application No. PCT/EP2019/086795 which was filed on Dec. 20, 2019, and which claims the priority of application LU 101075 filed on Dec. 28, 2018, the content of which (text, drawings and claims) are incorporated here by reference in its entirety.

### FIELD

The invention is directed to the field of human motion analysis, more particularly to a method and a system for analyzing the motion of a human body.

### BACKGROUND

The human body motion analysis is commonly used in sports to help athletes run more efficiently and to identify posture-related or movement-related problems in people with injuries. Generally, joint angles are used to track the location and orientation of body parts, to calculate the kinetics of the human body. Also, most labs dedicated to human body motion analysis have floor-mounted load transducers, which measure the ground reaction forces and moments.

In order to simplify the complex installation required for analyzing the human body motion (e.g. the gait analysis), an insole comprising pressure sensors has been conceived. In parallel, a glove with grip measurement sensors has been developed recently, improving ergonomics of hand related activities. These solutions have been found to be particularly useful. For instance, the simplicity of the insole fitted with pressure sensors and the development of smart devices such as smartphone make accessible such a technology to any users. For example, it could benefit the sportsman, people with mobility problem and people with any kind of physical, mobile or dynamic activities such as dancers to have their motions monitored outside a lab.

However, the analysis of the data measured assumes that the sensors are disposed in an “imaginary” flat median plane to the insole or the glove. In another word, it is a 2D approach. Also, it is supposed that the efforts are perpendicular to the median plane. Moreover, the known models cannot monitor efficiently the body motion when a person is on a slope or the deformations of the insole are significant. Concerning the hand related activities, the distance between the sensors plays an important role in the evaluation of the hand induced efforts. Known devices are silent on how to handle this.

### SUMMARY

The invention has for technical problem to provide a solution to at least one drawback of the above prior art. More specifically, the invention has for technical problem to provide a solution to improve the accuracy of the monitoring of the body motion.

For this purpose, the invention is directed to a method for monitoring a human body motion comprising the steps of: providing a garment with a surface comprising a plurality of stress sensors measuring at least one component of a stress tensor and generating output signals, the plurality of stress sensors being distributed over the surface and being adapted to face a portion of a human body; providing a means for detecting or recording 3D coordinates of each of the stress sensors; and providing an evaluation unit, the unit receiving the output signals of the plurality of stress sensors and the 3D coordinates; the unit being configured for: recording at least one stress value of the at least one component of the stress sensor measured for each stress sensor; estimating at least one stress value representative of a human body motion parameter in a point of the surface at least on the basis of at least two of the at least one stress values, corresponding respectively to the measurements of at least two of the stress sensors and at least two 3D coordinates, corresponding to the at least two of the stress sensors.

According to an exemplary embodiment, the at least one stress value in the point of the surface is estimated on the basis of all the at least one stress values of all stress sensors and all the 3D coordinates of each stress sensor.

According to an exemplary embodiment, the estimation is an interpolation, in various instances the interpolation is selected from the group consisting of natural neighbor interpolation, inverse distance weighted, trend surface interpolation, linear triangulation interpolation, spline interpolation, ordinary Kriging, simple Kriging, universal Kriging.

According to an exemplary embodiment, each of the stress sensors is a pressure sensor.

According to an exemplary embodiment, the at least one component of the stress tensor is a pressure.

According to an exemplary embodiment, the pressure sensor is defined by a normal vector perpendicular to a plane tangent to the pressure sensor measurement surface.

According to an exemplary embodiment, the means for detecting or recording 3D coordinates of each of the stress sensors is further configured for also detecting or recording a group of components of the normal vector of each stress sensor.

According to an exemplary embodiment, the evaluation unit is further configured for estimating at least one stress value representative of a human body motion parameter in a point of the surface also on the basis of at least two groups of components of the normal vector, corresponding to the at least two of the stress sensors.

According to an exemplary embodiment, each of the stress sensors is a multi-directional shear and normal force sensor.

According to an exemplary embodiment, the at least one component of the stress tensor comprises at least one normal stress component and at least one shear stress component.

According to an exemplary embodiment, the at least one normal stress component is defined by a normal vector perpendicular to a plane tangent to the surface at a given point, the at least one shear stress component is defined by a tangent vector within the plane, the at least one stress value comprises at least two stress values being respectively a pressure corresponding to the normal stress component associated with the normal vector and a shear stress corresponding to the at least one shear stress component associated with the tangent vector and normal vector.

According to an exemplary embodiment, the means for detecting or recording 3D coordinates of each of the stress sensors is further configured for also detecting or recording a group of components of the normal and tangential vectors of each stress sensor.

According to an exemplary embodiment, the evaluation unit is further configured for estimating at least one stress value representative of a human body motion parameter in a point of the surface also on the basis of at least two groups of components of the normal and tangential vectors, corresponding to the at least two of the stress sensors.

According to an exemplary embodiment, the garment is an article of footwear, a short, underpants or a glove.

According to an exemplary embodiment, the article of footwear is an insole, a sole of a shoe or a sock and the portion of a human body is a sole of a foot.

According to an exemplary embodiment, the step of providing an evaluation unit comprises the embedment of the evaluation unit within the garment.

According to an exemplary embodiment, the step of providing an evaluation unit comprises the embedment of the evaluation unit within a further garment selected from a group consisting of insole, sole of shoe, sock, short, underpants, glove, armband.

According to an exemplary embodiment, the point in the surface where the at least one stress value is estimated is different from any points corresponding to the 3D coordinates of the plurality of stress sensors.

According to an exemplary embodiment, the output signals corresponding to each of the at least one stress value are recorded for a period of time.

According to an exemplary embodiment, the evaluation unit determines key indicators for at least one cycle of a plurality of human body motion cycles.

According to an exemplary embodiment, acceptable values or ranges of values are defined for each key indicator and when one of the key indicators departs from its acceptable values or ranges of values, a warning signal is issued.

According to an exemplary embodiment, the evaluation unit is configured to detect a change of body motion based on the detected persistent variations prior to or during the detection of a key indicator departing from its acceptable values or ranges of values.

According to an exemplary embodiment, the acceptable values or ranges are preset or based on averaged or reference values of previous cycles.

According to an exemplary embodiment, the key indicators are averaged 3D coordinates of a weighted geometric center estimated for the at least one cycle, wherein the distribution of weight is based on at least one distribution of the at least one stress value.

According to an exemplary embodiment, the key indicators are averaged 3D coordinates of a pressure geometric center estimated for the at least one cycle.

According to an exemplary embodiment, the key indicators are further selected from the group consisting of: the maximum of the at least one stress value during the at least one cycle, the average of the at least one stress value over the at least one cycle, the duration of the at least one cycle, the point in time when the at least one stress value changes to exceed a reference value, the duration during which the at least one stress value exceeds the reference value, the integral of the at least one stress value over the duration of the at least one cycle, a linear combination thereof.

According to an exemplary embodiment, a plurality of human body motion cycles is a plurality of stride cycles.

According to an exemplary embodiment, a plurality of human body motion cycles is a plurality of hand motion cycles.

According to an exemplary embodiment, the values corresponding to the 3D coordinates of the stress sensors are calibrated after the garment being put onto the human body portion to take into account the actual positions of the sensors.

According to an exemplary embodiment, the values corresponding to the 3D coordinates of the stress sensors are based on measurements and/or a model.

According to an exemplary embodiment, values corresponding to the group of components of the normal vector of each stress sensor are estimated in an absolute referential.

According to an exemplary embodiment, the values corresponding to group of components of the normal vector of each stress sensor are based on measurements of an inertial sensor fitted into the garment, the evaluation unit or another wearable device such as a smart phone or on a combination of a digital elevation model and global positioning system coordinates estimated by the evaluation unit or another wearable device such as a smart phone.

The invention is also directed to a system for monitoring a human body motion comprising: a garment with a surface comprising a plurality of stress sensors measuring at least one component of the stress tensor and generating output signals, the plurality of stress sensors being distributed over the surface and being adapted to face a portion of a human body; a means for detecting or recording 3D coordinates of each of the stress sensors; an evaluation unit configured for receiving the output signals of the plurality of stress sensors and the 3D coordinates; the unit being configured for: recording at least one stress value of the at least one component of the stress sensor measured for each stress sensor; estimating at least one stress value representative of a human body motion parameter in a point of the surface on the basis of at least two of the at least one stress values, corresponding respectively to the measurements of at least two of the plurality of stress sensors and the 3D coordinates.

According to an exemplary embodiment, each of the stress sensors is a pressure sensor.

According to an exemplary embodiment, each of the pressure sensors is adapted to measure a pressure between 0 and 7 bars or between 0 and 3.5 bars.

According to an exemplary embodiment, each stress sensor is a multi-directional shear and normal force sensor.

According to an exemplary embodiment, the garment is an article of footwear, short, underpants or glove.

According to an exemplary embodiment, the article of footwear is an insole, a sole of a shoe or a sock and the portion of the human body is a sole of a foot.

According to an exemplary embodiment, the evaluation unit is integrated into the garment.

According to an exemplary embodiment, the evaluation unit is integrated into a further garment selected from a group consisting of insole, sole of shoe, sock, short, underpants, glove, armband.

The invention can capture the 3D effects that impact the measurements of human body motion parameters. For instance, the 3D forces applied to a portion of the body can be recorded. The precise positioning (3D coordinates) of the stress sensors is used for the monitoring. Also, the deformation of the garment and therefore the relative distances between the stress sensors can be supervised. The invention allows monitoring the gait for medical and sport purposes, wherein a practitioner or a trainer is directly informed of a change in the gait of patient or sportsman, respectively. The invention analyses the data recorded based on a learning algorithm, improving the quality of the data computed. Finally, the features of the invention allow analyzing the hand motions efficiently.

### DRAWINGS

Other features and advantages of the present invention will be readily understood from the following detailed description and drawings among them:

FIG. 1 represents a schematic view of an insole with stress sensors, in accordance with various embodiments of the invention.

FIG. 2 shows the insole with stress sensors with 3D coordinates and normal vectors, in accordance with various embodiments of the invention.

FIG. 3 depicts the variation in the distance between two stress sensors, in accordance with various embodiments of the invention.

FIG. 4 represents the insole with an absolute and relative coordinate systems, in accordance with various embodiments of the invention.

FIG. 5 shows the influence of an incline on the stress sensors measurements, in accordance with various embodiments of the invention.

FIG. 6 represents evolutions of stress sensors measurements, in accordance with various embodiments of the invention.

FIG. 7 represents the sum of a plurality of curves measured, in accordance with various embodiments of the invention.

FIG. 8 represents the results of an interpolation, in accordance with various embodiments of the invention.

FIG. 9 describes trajectories of the geometric center of pressure, in accordance with various embodiments of the invention.

FIG. 10 illustrates a glove with stress sensors, in accordance with various embodiments of the invention.

### DETAILED DESCRIPTION

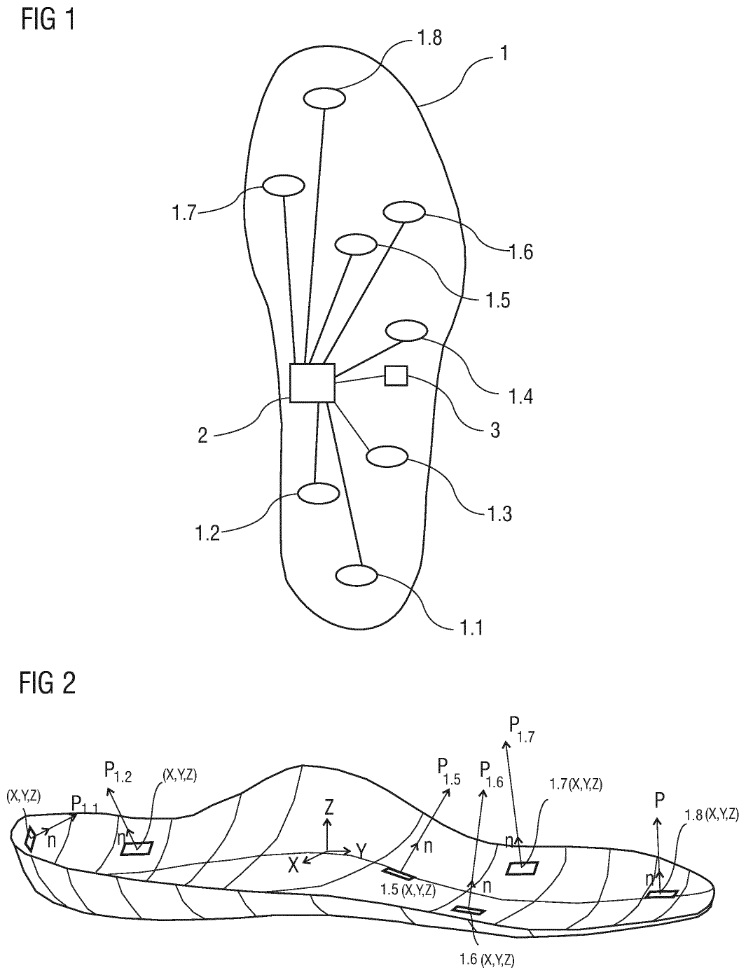

FIG. 1 depicts a schematic view of a garment 1, more precisely an insole 1 equipped with a plurality of stress sensors 1.1, 1.2, 1.3, 1.4, 1.5, 1.6, 1.7, 1.8 (1.1-1.8) adapted to measure at least one component of a stress tensor and generating output signals. The plurality of stress sensors 1.1-1.8 are distributed over the surface and are adapted to face a portion of a human body. The invention is not limited to an insole 1. Indeed, a plurality of sensors can be provided on a sock, a glove for instance. In the present case the insole 1 comprises 8 sensors 1.1-1.8. The number of sensors 1.1-1.8 can vary depending on the needs.

The garment 1 according to FIG. 1 can comprise an evaluation unit 2 for determining at least one human body parameter. The evaluation unit 2 receives output signals of the plurality of stress sensors 1.1-1.8. The evaluation unit 2 is at least configured for recording the output signals of each stress sensor 1.1-1.8 for a period of time. For this exemplary embodiment, the evaluation unit 2 is integrated in the insole 1 and connected to the sensors 1.1-1.8 via metallic wires. This arrangement allows short electric connections so that the user is not impeded. Alternatively, the evaluation unit 2 can be integrated into another garment such as a sock, a shirt, trousers, an armband for instance of the person monitored with the stress sensors 1.1-1.8. In various instances, the evaluation unit 2 is clipped to a sock or shoe, the evaluation unit 2 being connected to the sensors 1.1-1.8 of the insole 1 by wires. Alternatively, the communications between the plurality of sensors 1.1-1.8 and the evaluation unit 2 can be wireless. When the evaluation unit 2 is integrated into the other garment, the evaluation unit 2 can be a smart device such as a smartphone. Furthermore, the evaluation unit 2 can be a remote computing device used by a trainer or a doctor, the remote computing device being a laptop, a smartphone or a desktop for instance.

A means 3 for detecting or recording 3D coordinates of each of the stress sensors 1.1-1.8 is provided within the insole 1. Alternatively, the means 3 for detecting or recording 3D coordinates is integrated into the evaluation unit 2 or clipped to another garment 1 such as a sock or a shoe.

The garment 1 can comprise a source of energy such as a battery for the supply of current to the evaluation unit 2, the plurality of stress sensors 1.1-1.8 and the means 3 for detecting or recording 3D coordinates. In an alternative exemplary embodiment, the battery can be integrated into the evaluation unit 2, wherein the battery can be charged via an USB-port. The source of energy can be piezo energy using the compression on the insole 1 to generate electricity for the evaluation unit 2 and the plurality of sensors 1.1-1.8. Furthermore, the plurality of sensors 1.1-1.8 can be also piezo elements generating the electricity needed by the evaluation unit 2. The garment 1 can contain an external memory (not shown) to increase the memory capacity of the evaluation unit 2. The evaluation unit 2 can be connected (e.g. wireless) to a computer system (not represented), such as a smartphone or a remote server to store and analyze the recorded data.

FIG. 2 shows the insole 1 with a plurality of stress sensors 1.1-1.8. Each stress sensor 1.1-1.8 can be either a pressure sensor or a multi-directional shear and normal force sensor. The center of each sensor 1.1-1.8 can be defined by 3D coordinates. Another point of reference can be selected such as the center of the measurement surface. The means 3 for detecting or recording 3D coordinates can store values corresponding to the 3D coordinates of the stress sensors 1.1-1.8. The 3D coordinates of the stress sensors 1.1-1.8 are based on measurements and/or a model. For instance, the 3D coordinates can be constant and preset by a manufacturer. Alternatively, the 3D coordinates can be adjusted once the insole 1 is put, using a system that measures/calculates the position of the 3D coordinates in situ or remotely. To improve the monitoring of the body motion, it is necessary to consider variable 3D coordinates that change with the deformations of the garment 1, 101 (e.g. insole 1 or glove). For this purpose, it is proposed to have a model for the 3D coordinates that is predefined by the manufacturer for example. The model assumes that the 3D coordinates respect a general cyclic predefined pattern. For instance, a model for a reference cyclic posture of a foot and the corresponding deformation of an insole 1 is defined and stored in the means 3 for detecting or recording 3D coordinates. The model can be calibrated. The stress sensor measurements (e.g. pressure) are inputted in the model that computes the corresponding deformation of the insole 1. Therefore, the means 3 for detecting or recording 3D coordinates can compute the 3D coordinates on the basis of the sensor 1.1-1.8 measurements. Alternatively, the values of the 3D coordinates can be measured in real time using dedicated sensors that measure the relative distance between the stress sensors 1.1-1.8.

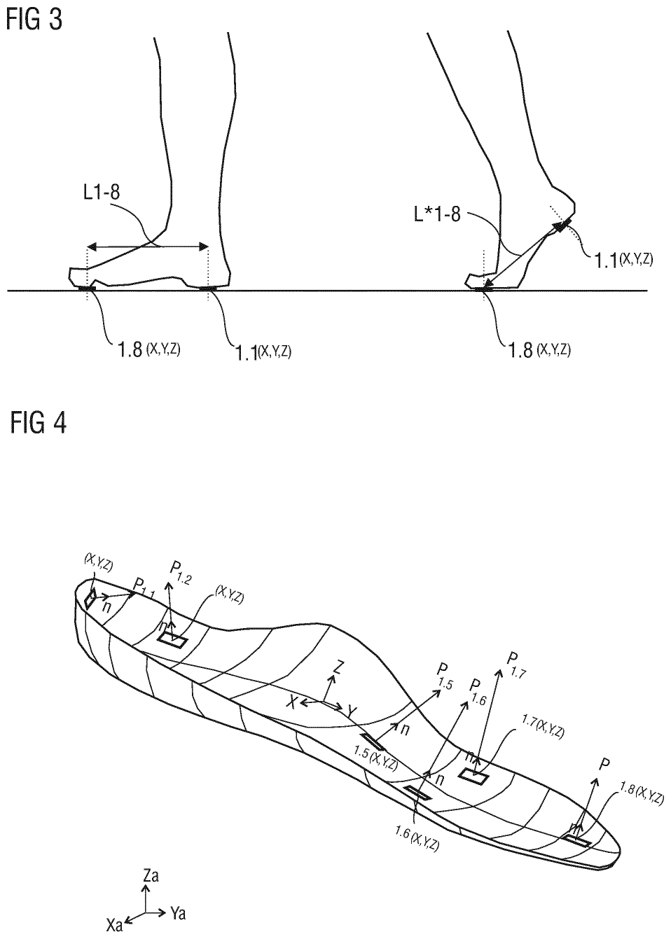

FIG. 3 illustrates that the distance between two sensors 1.1, 1.8 can vary during a stride. The necessity of monitoring of the variable positions of the sensors 1.1-1.8 depends on the application. For instance, the monitoring of an insole 1 can be performed assuming the 3D coordinates are constant, assuming that the deformations are not extreme. However, an application such as a glove needs information on the variable 3D coordinates of the sensors 1.1, 1.2, 1.3, 1.4, 1.6, 1.7, 1.8 to determine certain key indicators describing the motion of the part of the body in question.

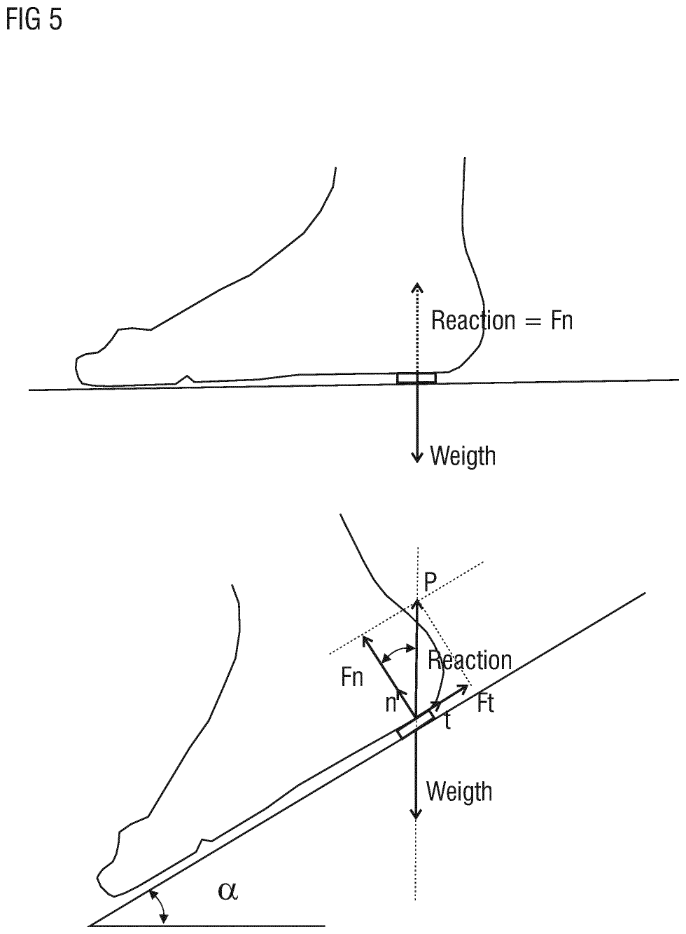

The position of the center can be defined with a relative coordinate system attached to the insole 1. Alternatively, the position of the center can present absolute coordinates, when the coordinate system is absolute, as shown in FIG. 4.

A stress sensor 1.1-1.8 can be either a pressure sensor 1.1-1.8 or a multi-directional shear and normal force sensor 1.1-1.8.

A pressure sensor 1.1-1.8 is defined by a normal vector perpendicular to a plane tangent to the pressure sensor measurement surface. The normal vector is defined by a group of 3 components (nx, ny, nz). As an alternative to a normal vector, the orientation of the plane can be defined by three points within the plane or a disk. The means 3 for detecting or recording 3D coordinates of each of the stress sensors 1.1-1.8 can be further configured for also detecting or recording a group of components of the normal vector of each pressure sensor 1.1-1.8.

A multi-directional shear and normal force sensor 1.1-1.8 is defined by a normal vector perpendicular to a plane tangent to the surface at a given point and by at least one tangent vector within the plane. The multi-directional shear and normal force sensor 1.1-1.8 allows improving the analysis of the body motion because it provides a tensorial description of the efforts. However, such a sensor 1.1-1.8 is complex. The means 3 for detecting or recording 3D coordinates of each of the stress sensors 1.1-1.8 can be further configured for also detecting or recording a group of components of the normal and the at least one tangential vectors of each stress sensor 1.1-1.8.

FIG. 5 shows a foot on an inclined surface and an insole 1 equipped with pressure sensors 1.1-1.8. When the foot is on a horizontal plane, the measure of the pressure recorded is representative of the weight. However, when the pressure is on an incline surface, the pressure recorded needs to be corrected by dividing the value of the pressure recorded by the cosine of the angle of the slope to get the value representing the real weight. It is therefore proposed to correct the measurement using the value of the slope via a digital elevation model (DEM) and global positioning system coordinates (GPS). The correction can be estimated by the evaluation unit 2 or another wearable device such as a smart phone. Also, knowing the slope allows to calculate a sheer component. Therefore, the combination of the slope and the pressure recorded can be used to reconstruct some or all components of the stress sensor. With this solution, it is not necessary to invest in a complex multi-directional shear and normal force sensor 1.1-1.8. The example of FIG. 5, shows the influence of the slope on the analysis of the body motion. Equally, an acceleration of the body or the insole 1 can be detected by the evaluation unit or another wearable device such as a smartphone. The measure or estimation of the acceleration can as well be used to reconstruct the component of the shear sensor.

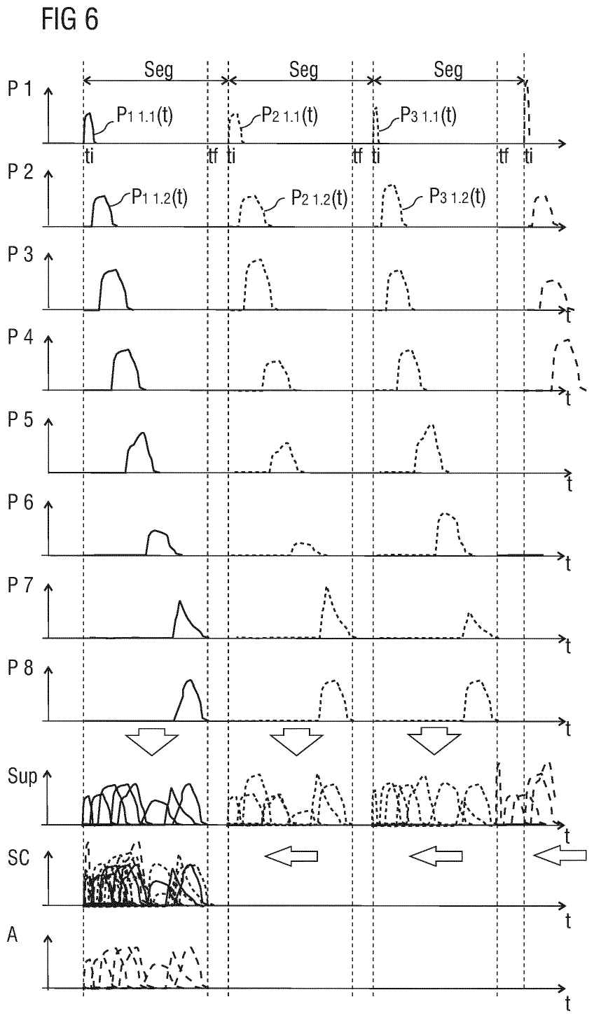

FIG. 6 illustrates the data recorded by the plurality of sensors 1.1-1.8 mounted into an insole 1, for instance. FIG. 6 shows pressure curves, wherein one graph P1-P8 is displayed for each sensor. The x-axis corresponds to the time, while the y-axis shows the pressure recorded. The pressure curves can be periodic or quasi periodic and show cycles corresponding to the strides. For the quasi periodic cycle, the period can change from one cycle to another. A stride cycle starts, for instance, during a walk or run, when a sensor positioned at the rear most position 1.1 of the insole 1 detects the contact of a shoe with the ground and ends when the same sensor 1.1 is pressed at the beginning of the next stride.

A stride cycle according to the invention also encompasses transient phase. For instance, a dancer stepping from the tip of a foot activating the most front sensor 1.8 to a position where the dancer steps on a heel activating the most rear sensor 1.1. In this case, two transient cycles take place. In the first one, only the most front sensor 1.8 is activated, while all other sensors remain inactivate. In the second one, the most rear sensor 1.1 is activated, while all other sensors remain inactivate. The definition of a stride can be adapted to the final use, e.g. running, climbing, walking, classical dance.

Key indicators KI are determined by first segmenting the measured curves as shown in FIG. 6, where an initial time ti is determined for each stride cycle. The initial time ti can be detected when the pressure sensor 1.1 positioned on the rear most position changes from a non-zero value to a zero value. The initial time ti for a stride can be defined as being the same for all pressures recorded as shown in FIG. 6. A segment Seg can be extracted for a given pressure curve. The segment Seg can start at the initial time ti and can end at the beginning of the initial time ti of the next stride cycle. This operation can be repeated for all the other output values, corresponding to the remaining N−1 segments Seg, N being the number of pressure sensors. All segments Seg can then be superposed on each other, as shown in FIG. 6 in graph Sup. The computation of the key indicators KI of the body motion such as the gait based on the superposed segmented curves serves as basis for the key indicator KI, which can be calculated for each stride. In order to reduce the fluctuation from one cycle to another, a collection of pressure curves can be grouped and then averaged over several stride cycles as shown in FIG. 6 in graphs SC and A, respectively.

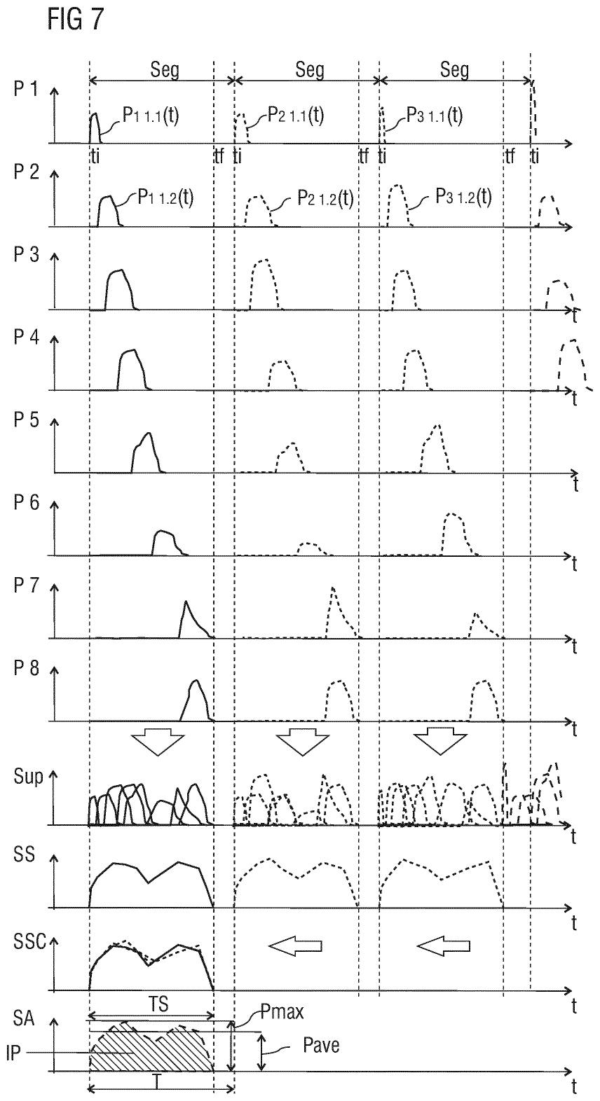

In an exemplary embodiment, as shown in FIG. 7, the plurality of pressure curves can be summed, grouped, and optionally averaged as shown on graphs SS, SSC and SA, respectively. The sum of the pressures curves is based on the following formula:

$${P}_{i} ( t ) = {\sum }_{k = 1}^{N} {P}_{i 1 . k} ( t )$$

where: Pᵢ (t) corresponds to the sum of the superposed pressures curves measured for each sensor for stride cycle i; P_{i 1.k}(t) corresponds to the segmented pressure curve recorded for the pressure sensor 1.k for the stride cycle i; N is the number of sensors.

The key indicators KI are selected from the group consisting of: the maximum pressure Pmax over a stride cycle, the average pressure Pave over the stride cycle, the duration T of the stride cycle, the point in time when the pressure changes from a non-zero value to a zero value, the duration during which the pressure curve is not equal to zero, the integral of the pressure IP over the duration of the stride cycle, a linear combination thereof, as shown in FIG. 7.

The point in time when the pressure changes from non-zero to a zero value on sensor 1.8 at the most front position of the article of footwear 1 can correspond to the final time tf. The duration between the initial time ti and the final time tf is the stance duration TS of the stride cycle. The stance duration TS of a stride cycle can be a further key indicator SK. The segment Seg can alternatively be defined as starting at the initial time ti and ending at the final time tf. Also, the swing duration for a cycle is the difference between the stride cycle duration T and the stance duration TS. The swing duration as well the ratio between the stance duration TS and the swing duration can be used as key indicators KI.

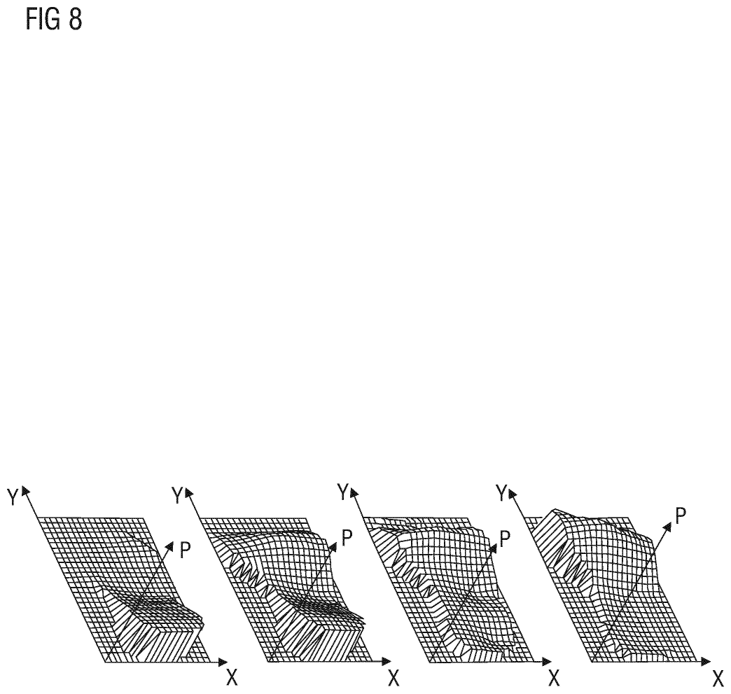

FIG. 8 shows the result of an interpolation of the pressure measurement over a stride. In various instances, the interpolation is selected from the group consisting of natural neighbor interpolation, inverse distance weighted, trend surface interpolation, linear triangulation interpolation, spline interpolation, ordinary Kriging, simple Kriging, universal Kriging. For the boundary limits of the interpolation model, it is generally assumed that the stress sensor components, such as the normal pressure, are equal to zero on the border of the insole 1.

In another exemplary embodiment, a position of the center of pressure is calculated at each moment in time based on a linear combination (weighted sum) of the superposed pressure curves of the plurality of pressure sensors. The position of the center of pressure can be determined with the following formulas:

$${x}_{G i} ( t ) = \frac{{\sum }_{k = 1}^{N} {x}_{1. k} {P}_{i 1. k} ( t )}{{\sum }_{k = 1}^{N} {P}_{i1 . k} ( t )} \\ {y}_{G i} ( t ) = \frac{{\sum }_{k = 1}^{N} {y}_{1. k} {P}_{i 1. k} ( t )}{{\sum }_{k = 1}^{N} {P}_{i 1. k} ( t )} \\ {z}_{G i} ( t ) = \frac{{\sum }_{k = 1}^{N} {z}_{1. k} {P}_{i 1. k} ( t )}{{\sum }_{k = 1}^{N} {P}_{i 1. k} ( t )}$$

where: x_{G i} (t) corresponds to the position of the geometric center of pressure G according to the x-axis for stride cycle i; y_{G i} (t) corresponds to the position of the geometric center of pressure G according to the y-axis for stride cycle i; z_{G ti} (t) corresponds to the position of the geometric center of pressure G according to the z-axis for stride cycle i; x_{1.k} corresponds to the position of the pressure sensor 1.k according to the x-axis; y_{1.k} corresponds to the position of the pressure sensor 1.k according to the y-axis; z_{1.k} corresponds to the position of the pressure sensor 1.k according to the z-axis; P_{i 1.k}(t) corresponds to the segmented pressure curve recorded for the pressure sensor 1.k for the stride cycle i; N is the number of sensors.

Also, the values of the interpolation method can be used, to estimate the position of the center of pressure. The position of the center of pressure can be determined with the following formulas:

$${x}_{G i} ( t ) = \frac{{\sum }_{l = 1}^{M} {x}_{l}^{*} {P}_{{i}_{l}}^{*} ( t )}{{\sum }_{l = 1}^{M} {P}_{{i}_{l}}^{*} ( t )} \\ {y}_{G i} ( t ) = \frac{{\sum }_{l = 1}^{M} {y}_{l}^{*} {P}_{{i}_{l}}^{*} ( t )}{{\sum }_{l = 1}^{M} {P}_{{i}_{l}}^{*} ( t )} \\ {z}_{G i} ( t ) = \frac{{\sum }_{l = 1}^{M} {z}_{l}^{*} {P}_{{i}_{l}}^{*} ( t )}{{\sum }_{l = 1}^{N} {P}_{{i}_{l}}^{*} ( t )} \qquad$$

where: x_{G i} (t) corresponds to the position of the geometric center of pressure G according to the x-axis for stride cycle i; y_{G i} (t) corresponds to the position of the geometric center of pressure G according to the y-axis for stride cycle i; z_{G i} (t) corresponds to the position of the geometric center of pressure G according to the z-axis for stride cycle i; xₗ* corresponds to the position of the interpolation node l according to the x-axis; yₗ* corresponds to the position of the interpolation node l according to the y-axis; zₗ* corresponds to the position of the interpolation node l according to the z-axis; Pᵢ_{ₗ}*(t) corresponds to the interpolated pressure estimated of for node l for the stride cycle i; M is the number of nodes of the interpolation method.

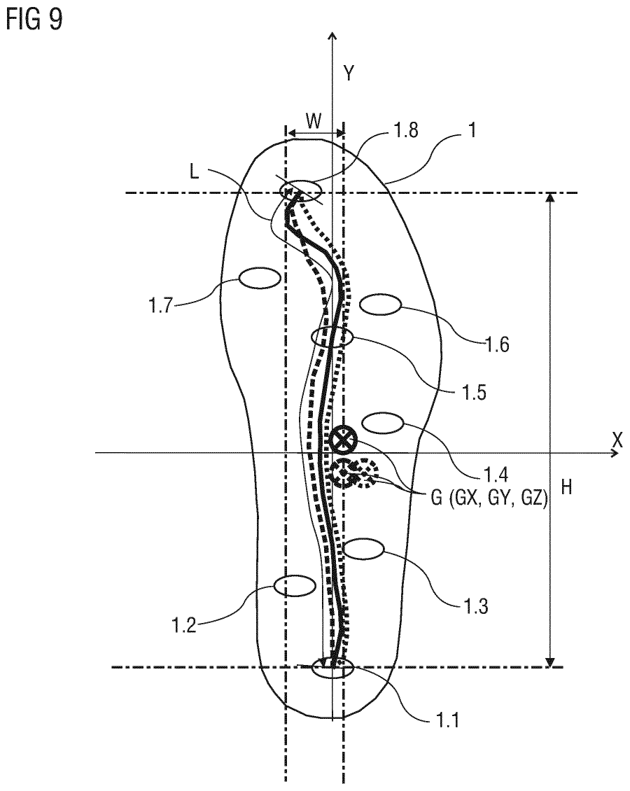

As shown on FIG. 9, the key indicators KI are determined on the basis of the trajectory of the geometric center G selected from the group consisting of: the distance L travelled by the position of the geometric center of pressure G per cycle i, the width W of the path traveled by the geometric center of pressure, the length H of the path covered by the geometric center of pressure, the elevation A (not shown) of the path traveled by the geometric center of pressure in z-axis direction. The length H of the path traveled can change when the person just rests on the heel activating a part of the pressure sensors. This occurs during a transition phase for instance. FIG. 9 shows the length H and width W determined for the continuous path line, which corresponds to a stride.

Also, the average center of pressure is a further key indicator KI and determined for each cycle with the coordinates (GX, GY, GZ), with the following formulas:

$$G X = \frac{{\int }_{ti}^{ti + T} {x}_{G i} ( t ) d t}{T}$$

$$GY = \frac{{\int }_{ti}^{ti + T} {y}_{G i} ( t ) d t}{T}$$

$$G Z = \frac{{\int }_{ti}^{ti + T} {z}_{G i} ( t ) d t}{T}$$

where: x_{G i} (t) corresponds to the position of the geometric center of pressure G according to the x-axis for stride cycle i; y_{G i} (t) corresponds to the position of the geometric center of pressure G according to the y-axis for stride cycle i; z_{G i} (t) corresponds to the position of the geometric center of pressure G according to the z-axis for stride cycle i; T is the duration of a stride cycle (T can change from one stride cycle to another). The trajectories of the geometric center of pressure can be averaged/smoothed to reduce the noise/fluctuation.

In an exemplary embodiment, the key indicators KI are monitored by the evaluation unit 2. For instance, acceptable values or ranges of values are defined for each key indicator KI. When one of the key indicators KI departs from its acceptable values or ranges of values, a signal is transmitted to the computing device so that a user is alerted.

Furthermore, the evaluation unit 2 is configured to generate a new key indicator KI based on the detected persistent variations prior to or during the detection of a key indicator KI departing from its acceptable values or ranges of values. For instance, an evaluation unit 2 is mounted on a shoe. In an initial phase, the pressures are recorded by an insole 1. Then, the user decides to wear a sock equipped with pressure sensors 1.1-1.8 replacing the insole 1. The transition from one article of footwear to another one implies a change in the coordinates of the pressure sensors. This requires an adaptation of the computation of key indicators KI, because the relative distances between the sensors 1.1-1.8 are altered.

The introduction of the key indicators KI allows a significant reduction in the amount of information, simplifying the management of the memory and reducing the required memory capacity.

Equally, the evaluation unit 2 is configured to detect a change of body motion, on the basis of a key indicator departing from its acceptable values or ranges of values. For instance, a user abnormal gait can be detected following an injury of the user. This abnormality can generate an incident that is sent to a practitioner so that further actions can be taken. Equally, the acceptable values or ranges can be preset or be based on averages of previous cycle. For instance, a person climbing a mountain can be monitored. The comparison of the key indicators KI between the beginning of the climb and the end for instance can reflect the tiredness of climber and/or the degree of the slope of the climb. Also, the comparison to the average of previous cycle, allows filtering for slow variations resulting for extrinsic perturbation and not resulting from a change in the gait.

The plurality of stress sensors such as pressure sensors 1.1-1.8 are adapted to measure a pressure between 0.1 and 7 bars. The detection of a passage from non-zero value to a zero value or from a zero value to a non-zero value could be based on a minimal pressure detection threshold, in various instances 0.1 bar. The minimal pressure detection threshold can correspond to the resolution of the pressure measurement, therefore amounting to 0.1 bar.

The key indicators KI can also be treated to represent relative values. For instance, the stand duration TS can be divided by the stride cycle T and the corresponding ratio can be used as a key indicator KI. In the case of a pair of insoles, the relative values allow comparing easily the key indicators KI of the two insoles.

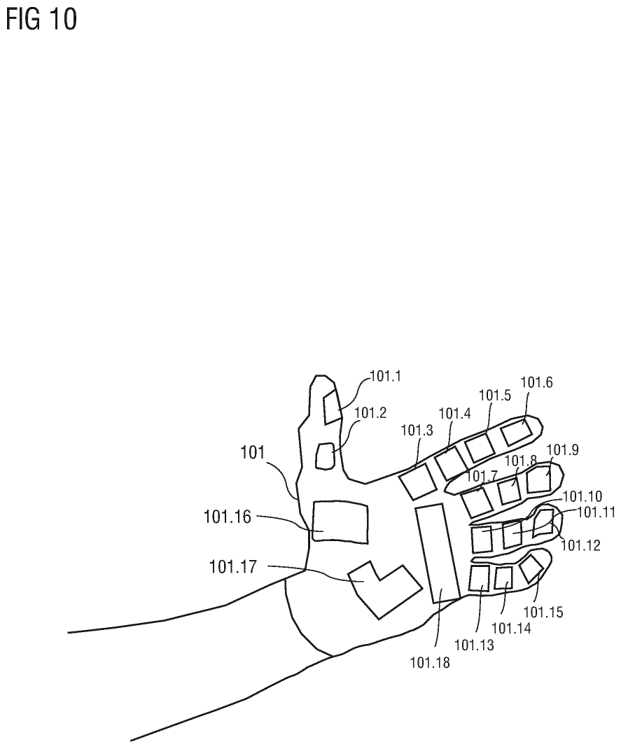

FIG. 10 shows another exemplary embodiment of the invention where a glove 101 comprises several stress sensors 101.1-101.18 more particularly several pressure sensors 101.1-101.18. The pressure measurements and the 3D coordinates of the pressure sensors are recorded. The corresponding values are used to interpolate the pressure distribution or key indicators KI in the same way as it was presented for the insole 1. The evaluation unit 2 (not shown) can be integrated into an armband (not shown). The pressure sensors 101.1-101.18 for the glove 101 are adapted to measure a pressure between 0.1 and 3.5 bars. A typical application is for instance the monitoring of a worker using a tool and performing a repetitive motion. The monitoring of the pressure helps an ergonomist to advise the worker. Indeed, the readings of the curves show whether the pressures of frequencies exceed acceptable values. In such a case, the ergonomist would advise to change the tool, for instance. Even if the exemplary embodiments are presented for an insole 1 or a glove 101, the invention can be generalized to any garment.

1.-40. (canceled)

41. A method for monitoring a human body motion comprising the steps of:
providing a garment with a surface adapted to face a portion of a human body and stress sensors distributed over the surface;
providing an evaluation unit;
measuring, by each of the stress sensors, at least one component of a stress tensor and generating output signals corresponding to the at least one component of a stress tensor;
one of detecting or recording 3D coordinates of each of the stress sensors; and
receiving, by the evaluation unit, the output signals of the stress sensors and the 3D coordinates of each of the stress sensors, wherein the evaluation unit is configured for:
recording for each stress sensor at least one stress value of the at least one component of the stress tensor;
estimating a human body motion parameter in a point of the surface based on at least two of the at least one stress values, corresponding respectively to the measurements of at least two of the stress sensors and at least two 3D coordinates, corresponding to the at least two stress sensors.

42. The method according to claim 41, wherein the point in the surface where the at least one stress value is estimated has a different location than any of the 3D coordinates of the plurality of stress sensors.

43. The method according to claim 41, wherein the human body motion parameter in a point of the surface is estimated based on at least one stress value of all the stress sensors and all their respective 3D coordinates.

44. The method according to claim 41, wherein estimating the human body parameter comprises an interpolation selected from the group consisting of: natural neighbor interpolation, inverse distance weighted, trend surface interpolation, linear triangulation interpolation, spline interpolation, ordinary Kriging, simple Kriging, and universal Kriging.

45. The method according to claim 41, wherein the stress sensors are pressure sensor and the at least one component of the stress tensor is a pressure.

46. The method according to claim 45, wherein the pressure is measured perpendicularly to a plane tangent to the surface at the respective 3D coordinates of each stress sensor.

47. The method according to claim 46, wherein detecting or recording 3D coordinates comprises detecting or recording components of a normal vector that is perpendicular to the plane tangent to the surface at the respective 3D coordinates of each stress sensor.

48. The method according to claim 47, wherein the evaluation unit is further configured to estimate the human body motion parameter based on the components of the normal vectors corresponding to the at least two of the stress sensors.

49. The method according to claim 41, wherein the stress sensors are each a multi-directional shear and normal force sensor.

50. The method according to claim 49, wherein recording for each stress sensor at least one stress value of the at least one component of the stress tensor consists of recording for each stress sensor at least two stress values of the at least two components of the stress tensor, the at least two components of the stress tensor being made of at least one normal stress component and at least one shear stress component, wherein the at least one normal stress component is a stress component measured perpendicularly to a plane tangent to the surface at a given point and the at least one shear stress component is measured within the plane tangent to the surface.

51. The method according to claim 41, wherein the one of detecting or recording 3D coordinates of each of the stress sensors comprises one of detecting or recording the position and orientation of each stress sensor.

52. The method according to claim 41, wherein the garment is one of: an article of footwear, a short, underpants and a glove, and wherein providing an evaluation unit comprises embedding the evaluation unit within the garment.

53. The method according to claim 41, further comprising providing a further garment selected from the group: insole, sole of shoe, sock, short, underpants, glove, and armband, and wherein providing an evaluation unit comprises embedding the evaluation unit within the further garment.

54. The method according to claim 41, wherein the evaluation unit determines key indicators, for at least one cycle of a plurality of stride cycles, wherein one of acceptable values or ranges of values are defined for each key indicator and when one of the key indicator departs from its one of acceptable values or ranges of values, a warning signal is issued, wherein the one of acceptable values or ranges of values are one of preset or based on averaged values of previous cycles.

55. The method according to claim 54, wherein a distribution of weight on the surface is determined based on the at least one stress value, and a key indicator is an averaged 3D coordinate of a weighted geometric center, based on the distribution of weight during the at least one cycle.

56. The method according to claim 54, wherein the key indicators are averaged 3D coordinates of a pressure geometric center estimated for the at least one cycle.

57. The method according to claim 41, further comprising putting the garment onto the human body portion followed by calibrating the 3D coordinates of the stress sensors.

58. The method according to claim 41, wherein the 3D coordinates of the stress sensors are based on at least one of measurements and a model.

59. The method according to claim 34, wherein each of the stress sensors is a pressure sensor adapted to measure a pressure between 0 and 100 psi.

60. A system for monitoring a human body motion, said system comprising:
a garment with a surface adapted to face a portion of a human body and stress sensors distributed over the surface, the stress sensors measuring a stress tensor and generating output signals;
an evaluation unit configured for receiving the output signals from the stress sensors, the unit being configured for:
recording at least one stress value of the stress tensor measured by each stress sensor;
estimating a human body motion parameter in a point of the surface based on at least two of the at least one stress values, corresponding respectively to the measurements of at least two of the stress sensors and at least two 3D coordinates corresponding to the location of the at least two the stress sensors.
